(19) United States
(12) Patent Application Publication
(10) Pub. No.: US 2016/0008634 A1
(43) Pub. Date: Jan. 14, 2016

(54) IMAGE-GUIDED FOCUSED ULTRASOUND ABLATION

(71) Applicant: Arapeen Medical, LLC, Jeremy Ranch, UT (US)

(72) Inventors: Allison PAYNE, Salt Lake City, UT (US); Dennis PARKER, Centerville, UT (US); Rock HADLEY, Centerville, UT (US); Nassir MARROUCHE, Park City, UT (US); David JENKINS, Salt Lake City, UT (US); Jesse CROWNE, Salt Lake City, UT (US)

(21) Appl. No.: 14/795,600

(22) Filed: Jul. 9, 2015

Related U.S. Application Data
(60) Provisional application No. 62/022,620, filed on Jul. 9, 2014.

(51) Int. Cl. A61N 7/00; A61B 5/055

(52) U.S. Cl. CPC A61N 7/00; A61B 5/055

(57) ABSTRACT

MRI-guided high-intensity focused ultrasound (“HIFU”) therapy can be used to ablate sympathetic nerves near the renal arteries. Such therapy may employ a HIFU device that includes an ultrasound transducer and an expandable reservoir that includes a first surface configured to conform to a surface anatomy of a patient and a port in fluid communication with a fluid source. The expandable reservoir can be configured to change a size in a dimension by inflating or deflating the expandable reservoir. Such a device may be used in connection with an MRI device that includes a support portion configured to conform to the surface anatomy of the patient and defining a first window configured to receive the ultrasound transducer for targeting tissue. The MRI device can include radiofrequency coil arrays embedded throughout to the support portion to monitor the therapy.

### RELATED APPLICATIONS

This application claims the priority benefit of U.S. Provisional Application No. 62/022,620, filed Jul. 9, 2014, the entirety of which is hereby incorporated herein by reference.

### FIELD

The subject technology relates to ablation treatment by ultrasound energy, including by ablation of renal nerves.

### BACKGROUND

Hypertension represents a critical health challenge for millions of people, affecting 74.5 million adults in the United States and costing approximately $76.6 billion when considering direct and indirect costs. Despite the availability of numerous pharmaceutical agents, roughly 40% of patients have uncontrolled hypertension. Since increased age and obesity are two of the most significant risk factors for hypertension, these numbers are expected to drastically increase making the treatment of hypertension a significant public health challenge. While there are many with uncontrolled hypertension, this is usually due to lack of patient adherence to the physician prescribed treatment, or inadequate treatment. However, approximately 10% of the patient population who are currently taking 3 medications or more continue to have persistent high blood pressure and are identified with resistant hypertension.

Kidneys play a major role in the chronic regulation of blood pressure, mainly through the regulation of sodium and water excretion. Renal sympathetic nerves are key in initiating and maintaining systemic hypertension and regulate several renal functions that are believed to contribute to hypertension including renal hemodynamics, renal tubular absorption of sodium and water, norepinephrine release and the renin secretion rate. Indeed, before effective pharmaceutical treatments were available, the surgical removal of these nerves was used as an effective treatment for hypertension, although this procedure had high morbidity rates. The proposed use of a non-invasive renal denervation procedure has the potential to produce the same efficacy without the high morbidity rates.

Many traditional renal denervation techniques apply energy with a catheter-based technique increasing procedural risk and restricting the eligibility of potential candidates. High intensity focused ultrasound (HIFU) is a completely non-invasive energy delivery technology that can deliver energy deep into tissue and can facilitate change on a cellular level through both thermal and mechanical effects. Additionally, nerve conduction can be temporarily or permanently suspended through application of HIFU. Applying HIFU under MRI guidance (MRgHIFU) provides accurate visualization of the treatment region and real-time monitoring of the energy delivery allowing for both treatment monitoring and efficacy assessment.

### SUMMARY

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 15, clause 23, or clause 27. The other clauses can be presented in a similar manner.

Clause 1. An apparatus for imaging a patient during an ultrasound treatment, comprising:

a support portion configured to conform to a surface anatomy of a patient and defining a first window configured to receive an ultrasound transducer for targeting a first region on a first side of the patient; and

radiofrequency coil arrays attached to the support portion.

Clause 2. The apparatus of clause 1, wherein the support portion further defines a second window configured to receive the ultrasound transducer for targeting a second region on a second side, opposite the first side, of the patient.

Clause 3. The apparatus of clause 2, further comprising a modular insert configured to fit within the first window and/or the second window.

Clause 4. The apparatus of clause 2, wherein the first side is a first lateral side and wherein the second side is a second lateral side.

Clause 5. The apparatus of clause 2, wherein the first side is an anterior side and wherein the second side is a posterior side.

Clause 6. The apparatus of clause 1, wherein the support portion comprises:

a support portion defining a first receptacle on the first side and a second receptacle on the second side, opposite the first side, of the patient;

a treatment portion, defining the first window, configured to be placed in the first receptacle; and

a modular portion configured to be placed in the second receptacle.

Clause 7. The apparatus of clause 6, wherein, during a procedure in which the support portion remains stationary relative to the patient, the treatment portion is configured to be moved to the second receptacle and the modular portion is configured to be moved to the first receptacle.

Clause 8. The apparatus of clause 6, the radiofrequency coil arrays are distributed among the support portion, the treatment portion, and the modular portion.

Clause 9. The apparatus of clause 1, wherein the support portion is configured to wrap entirely around a circumference of a body of the patient while conforming to the surface anatomy.

Clause 10. The apparatus of clause 1, wherein the first window is configured to be aligned on an anterior side of the patient when the support portion conforms to the surface anatomy.

Clause 11. The apparatus of clause 1, wherein the first window is configured to be aligned on a posterior side of the patient when the support portion conforms to the surface anatomy.

Clause 12. The apparatus of clause 1, wherein the first window comprises an echolucent material.

Clause 13. The apparatus of clause 1, wherein the surface anatomy is an abdomen of the patient, the first region includes a first renal nerve of the patient, and the second region includes a second renal nerve of the patient.

Clause 14. The apparatus of clause 1, further comprising the ultrasound transducer.

Clause 15. A method of imaging a patient during an ultrasound treatment, comprising:

applying a support portion to conform to a surface anatomy of a patient;

aligning a focal region of an ultrasound transducer with a first region on a first side of the patient;

ablating tissue within the first region with ultrasound energy from the transducer and through a first window of the support portion; and

obtaining an image of the first region with radiofrequency coil arrays attached to the support portion.

Clause 16. The method of clause 15, further comprising:

aligning the focal region of the ultrasound transducer with a second region on a second side of the patient;

ablating tissue within the second region with ultrasound energy from the transducer and through a second window of the support portion; and

obtaining an image of the second region with the radiofrequency coil arrays.

Clause 17. The method of clause 16, further comprising placing a modular portion, containing some of the radiofrequency coil arrays, in the second window.

Clause 18. The method of clause 16, further comprising placing a modular portion, containing some of the radiofrequency coil arrays, in the first window.

Clause 19. The apparatus of clause 16, wherein the surface anatomy is an abdomen of the patient, the first region includes a first renal nerve of the patient, and the second region includes a second renal nerve of the patient.

Clause 20. The method of clause 15, wherein the support portion remains stationary relative to the patient during the aligning.

Clause 21. The method of clause 15, wherein the applying comprises aligning the first window on an anterior side of the patient.

Clause 22. The method of clause 15, wherein the applying comprises aligning the first window on a posterior side of the patient.

Clause 23. An apparatus for performing an ultrasound treatment, comprising:

an ultrasound transducer having a focal length at which a focal region is formed; and

an expandable reservoir comprising:

a first surface configured to conform to a surface anatomy of a patient; and

a port in fluid communication with a fluid source;

wherein the expandable reservoir is configured to change a size in a dimension by inflating or deflating the expandable reservoir.

Clause 24. The apparatus of clause 23, wherein the size of the expandable reservoir determines a location of the focal region within the patient when the expandable reservoir conforms to the surface anatomy.

Clause 25. The apparatus of clause 23, wherein the expandable reservoir abuts the ultrasound transducer.

Clause 26. The apparatus of clause 23, wherein the focal length is fixed relative to the transducer.

Clause 27. A method for performing an ultrasound treatment, comprising:

applying a first surface of an expandable reservoir to a patient to conform to a surface anatomy of the patient;

positioning, within the patient, a focal region of an ultrasound transducer connected to the expandable reservoir by inflating or deflating the expandable reservoir.

Clause 28. The method of clause 27, wherein the positioning comprises injecting fluid into the expandable reservoir and/or aspirating fluid out of the expandable reservoir.

Clause 29. The apparatus of clause 27, wherein the surface anatomy is an abdomen of the patient and the positioning comprises aligning the focal region with a renal nerve of the patient.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 shows a view of portions of a patient's anatomy.

FIG. 2 shows a view of portions of a patient's anatomy.

FIG. 3 shows a view of an array of transducers focused on a renal nerve (not to scale), according to some embodiments of the present disclosure, according to some embodiments of the subject technology.

FIG. 4 shows a view of portions of a patient's anatomy that can be targeted during a therapeutic procedure, according to some embodiments of the subject technology.

FIG. 5 shows a view of HIFU devices applied to a portion of a patient's anatomy, being shown in cross-section, according to some embodiments of the subject technology.

FIGS. 6A and 6B show front perspective views of an ablation system, according to some embodiments of the subject technology.

FIGS. 6C and 6D show rear perspective views of an ablation system with module components, according to some embodiments of the subject technology.

FIGS. 6E and 6F show sectional views of an ablation system applied to a portion of a patient's anatomy, according to some embodiments of the subject technology.

FIG. 7 shows a block diagram of a MRI-guided ultrasound system, according to some embodiments of the subject technology.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

According to some embodiments, an apparatus and method for using MRI-guided focused ultrasound to ablate sympathetic nerves near the renal arteries may be employed to allow reduction of blood pressure. According to some embodiments, provided are devices and procedures to focus high intensity, ultrasonic acoustic waves into the tissue. High-intensity focused ultrasound (“HIFU”) is a highly precise medical procedure using high-intensity focused ultrasound to heat and destroy tissue.

As an acoustic wave propagates through the tissue, at least part of it is absorbed and converted to heat. With focused beams, a very small focus can be achieved deep in tissues. When hot enough, the tissue is thermally coagulated. By focusing at more than one place or by scanning the focus, a volume of tissue can be thermally ablated. In HIFU therapy, ultrasound beams are focused on targeted tissue, and due to the significant energy deposition at the focus, temperature within the tissue rises, destroying the diseased tissue by coagulation necrosis. Each sonication of the beams treats a precisely defined portion of the targeted tissue.

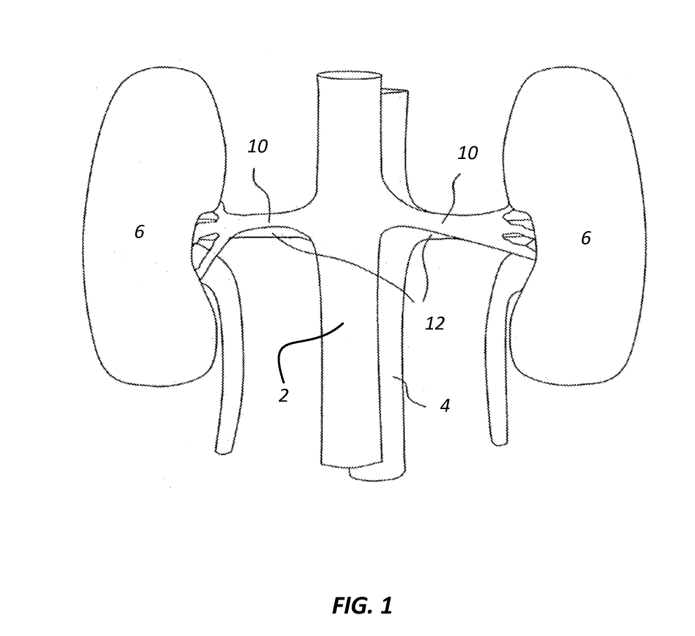

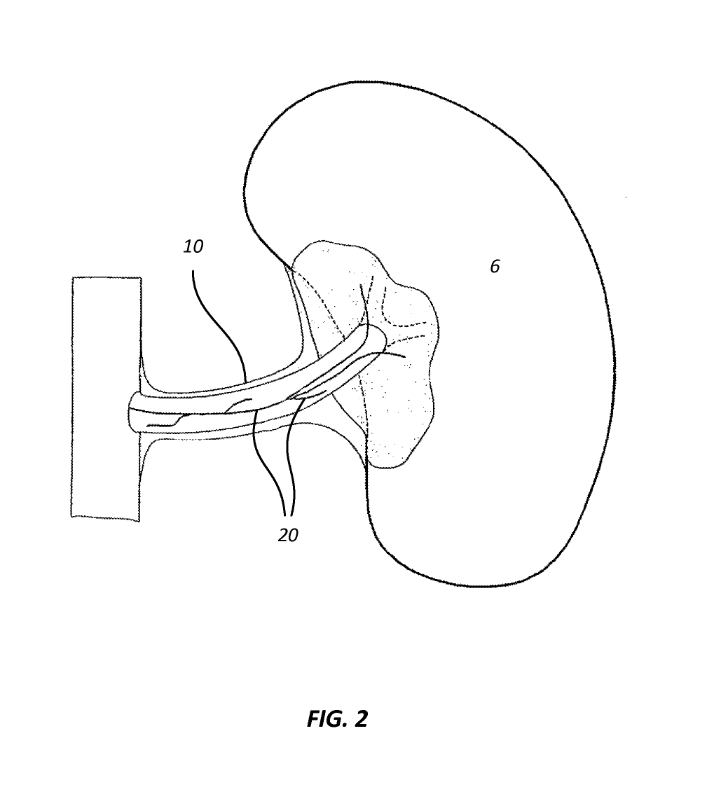

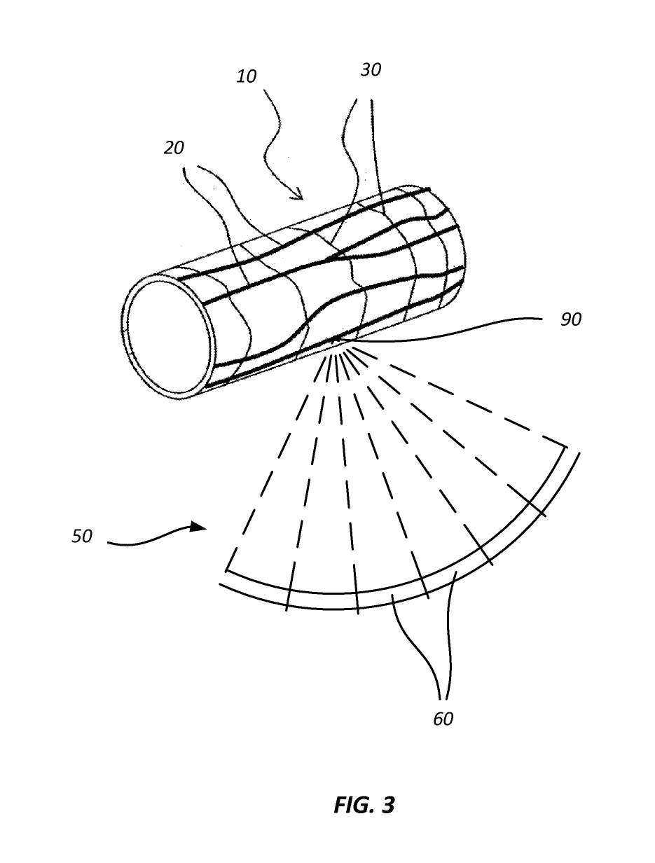

With reference now to FIG. 1, the human renal anatomy includes kidneys 6 that are supplied with oxygenated blood by renal arteries 10, which are connected to the heart by the abdominal aorta 2. Deoxygenated blood flows from the kidneys to the heart via renal veins 12 and the inferior vena cava 4. FIGS. 2-3 illustrate portions of the renal anatomy, including renal nerves 20 extending longitudinally along a lengthwise dimension of the renal artery 10 generally within the adventitia of the artery. The renal artery 10 has smooth muscle cells 30 that surround the arterial circumference and spiral around the artery.

According to some embodiments, as shown in FIG. 3, a HIFU device 50 may comprise one or more transducers 60 (e.g., 60a and 60b) for emitting ultrasound energy. Where a HIFU device 50 comprises an array of transducers 60 is provided, constituent transducers 60 of the array may be directed to converge generally at a focal region 90. The focal region 90 may be determined by position of the constituent transducers 60 relative to each other or position of the array relative to a target site.

According to some embodiments, as shown in FIG. 4, a HIFU device 50 can target tissue within one or both of two lateral sides of the patient. Transducers 60 of a HIFU device 50 can transmit sonic energy through one or both of two windows 94 (e.g., 94a and 94b) to focus on the target site. According to some embodiments, windows 94 can be located on an anterior side of the patient (e.g., with the patient in supine position) or a posterior side of the patient (e.g., with the patient in proposition). A procedure involving a HIFU device 50 can access one or more target sites (e.g., of the renal artery 10) through windows 94 located on a left anterior side of the patient, a right anterior side of the patient, a left posterior side of the patient, and/or a right posterior side of the patient. Target sites can be accessed through any combination of such windows 94 in sequence and/or simultaneously. For example, as shown in FIG. 4, a HIFU device 50 can access regions at or near one or more renal arteries 10 through a window 94 above a pelvis 16 of a patient and below ribs 14 of the patient.

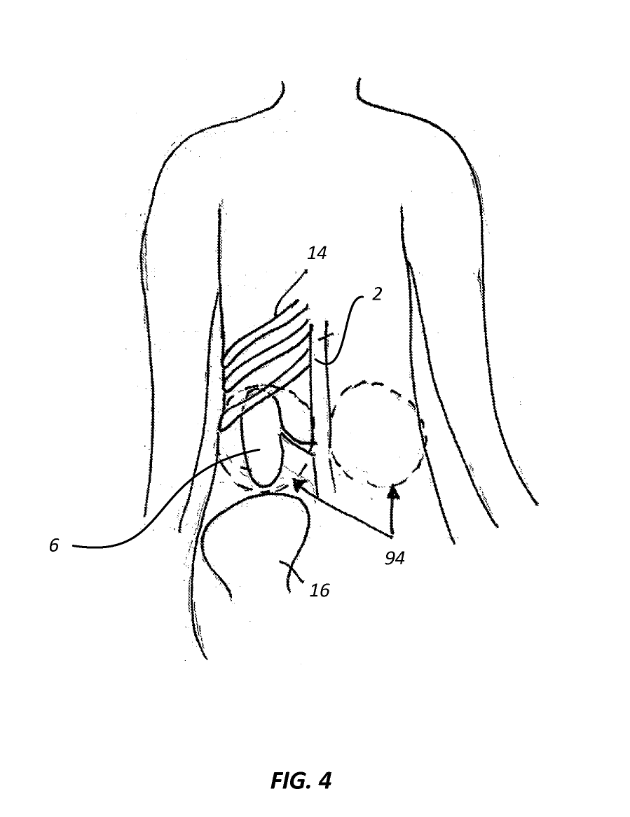

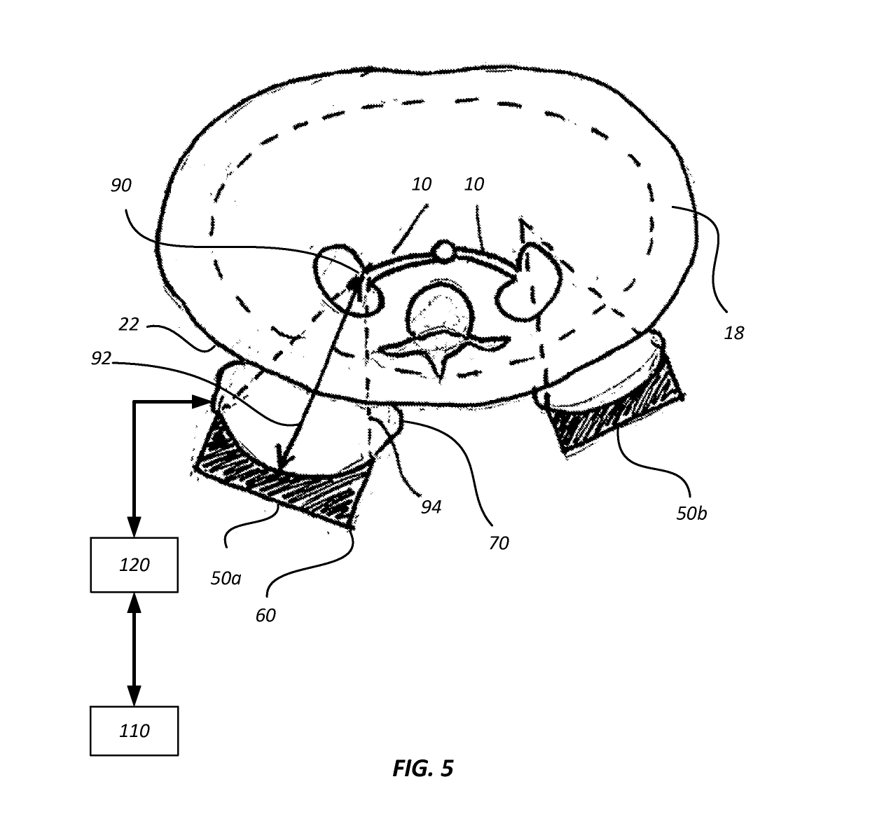

According to some embodiments, as shown in FIG. 5, a HIFU device 50 (e.g., 50a and 50b) can include an ultrasound transducer 60 and an expandable reservoir 70. The ultrasound transducer 60 can have a focal length 92 corresponding to a distance to a focal region 90 at which ultrasound beams converge. The ultrasound beams can travel through a window 94. From an ultrasound transducer 60, the beams can travel through a fluid filled expandable reservoir 70, a coupling fluid, a surface anatomy 22 (e.g., skin) of the patient, tissue within the patient (e.g., subcutaneous fat layers 18), and to a target site at the focal region 90. The HIFU device 50 can be coupled to the surface anatomy 22 with an appropriate coupling fluid such as degassed water. For example, a coupling fluid may be provided where the expandable reservoir 70 contacts the surface anatomy 22 of the patient. The coupling fluid can provide an echolucent transmission medium from the HIFU device 50 to the patient (e.g., at the window 94).

According to some embodiments, the focal length 92 of an ultrasound transducer 60 can be fixed or fixable, such that the focal length 92 does not change during a procedure. As such, the location of the focal region 90 can be adjusted by mechanical repositioning of the ultrasound transducer 60. According to some embodiments, an ultrasound transducer 60 can be coupled to an expandable reservoir 70 containing a fluid. The expandable reservoir 70 can increase in size and at least one dimension to adjust the position of the ultrasound transducer 60. For example, the expandable reservoir 70 can be inflated or deflated to move the ultrasound transducer 60 relative to the patient. According to some embodiments, adjustment of the size of the expandable reservoir 70 moves the ultrasound transducer 60 and/or the focal region 90 toward or away from a particular region within a patient.

According to some embodiments, as shown in FIG. 5, the expandable reservoir 70 includes a port in fluid communication with a fluid source 110. A fluid of the fluid source 110 can be conducive to ultrasound procedures by providing transmission of ultrasound beams. According to some embodiments, a pump 120 can facilitate inflation and/or deflation of the expandable reservoir 70 by moving the fluid from the fluid source 110 to the expandable reservoir 70, or vice versa.

According to some embodiments, the expandable reservoir 70 may include a wall of a flexible material, such as a polymer or an elastomer. The expandable reservoir 70 may be formed, in part or in whole, by one or more resiliently expandable materials. According to some embodiments, the wall of the expandable reservoir 70 may also include a rigid material to reduce expansion and/or contraction of the expandable reservoir 70 in some directions. For example, lateral sides of the expandable reservoir 70 not adjacent to the surface anatomy 22 may be of a rigid material, relative to a flexible material of the expandable reservoir 70, to cause inflation and deflation to be focused at an end of the expandable reservoir 70 that contacts the surface anatomy 22. According to some embodiments, relative rigidity of portions of the expandable reservoir 70 may be achieved by varying the thickness of a wall of the expandable reservoir 70.

According to some embodiments, a plurality of expandable reservoirs 70 can be provided to a single ultrasound transducer 60. Each of the plurality of expandable reservoirs 70 can contact the surface anatomy 22 of the patient, such that each of the expandable reservoirs 70 defines a distance between a portion of the ultrasound transducer 60 and the surface anatomy 22 of the patient. Independent adjustment of the plurality of expandable reservoirs 70 can move the ultrasound transducer 60 in a plurality of dimensions. For example, adjustment of the plurality of expandable reservoirs 70 can move the ultrasound transducer 60 toward, away from, and/or laterally across the surface anatomy 22 to position the focal region 90 at a target region within a patient. By further example, independent adjustment of the plurality of expandable reservoirs 70 can rotationally adjust the orientation of the ultrasound transducer 60 relative to the surface anatomy 22 to position the focal region 90 at a target region within a patient. For example, three, four, or a greater number of expandable reservoirs 70 can be provided between the ultrasound transducer 60 and the surface anatomy 22 of the patient, such that independent adjustment of the plurality of expandable reservoirs 70 can controllably and predictably move the focal region 90 of the ultrasound transducer 60 in three-dimensional space. By further example, by controlling the orientation of the ultrasound transducer 60 (e.g., angular coordinate, polar angle, etc.) and the distance between the ultrasound transducer 60 and the surface anatomy 22 (radial coordinate, radius, etc.), a control system can position the focal region 90 according to parameters of a polar coordinate system.

Remote, localized tissue ablation using HIFU can include sudden thermal necrosis due mainly to the absorption of ultrasound energy. The temperatures thus induced (e.g., about 60-80° C.) can produce irreversible changes in the targets. Target temperature thresholds may be any temperature above body temperature. For example, target temperature thresholds may include temperatures equal to or greater than 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.

Therapeutic ultrasound may be provided as minimally invasive or non-invasive. Further, it may be provided transcutaneously, subcutaneously, intravascularly, inter alia. In addition to the above, an ultrasound beam can also be focused geometrically, for example with a lens, with a spherically curved transducer, or electronically, by adjusting the relative phases of elements in an array of transducers (a “phased array”). By dynamically adjusting the electronic signals to the elements of a phased array, the beam can be steered to different locations, and aberrations due to tissue structures can be corrected.

Incorporated herein by reference are the following US applications and/or publications containing further teachings regarding HIFU therapy: US Pub. No. 2007/0167773, published on Jul. 19, 2007; U.S. Pat. No. 5,769,790, issued on Jun. 23, 1998; US Pub. No. 2008/0312561, published on Dec. 18, 2008.

According to some embodiments, systems and methods for imaging tissue using magnetic resonance imaging (“MRI”) techniques may be used. Thermal surgery guided by MRI systems and procedures can be used to selectively destroy tissue in a patient with localized heating, without adversely affecting tissue that is to remain substantially unaffected by the procedure. According to some embodiments, an MRI device 150 with RF coils can be designed to receive signals from tissues such as muscle, glandular tissue, and fat (among other tissues). An MRI pulse sequence is provided to obtain images that measure temperature in the tissues.

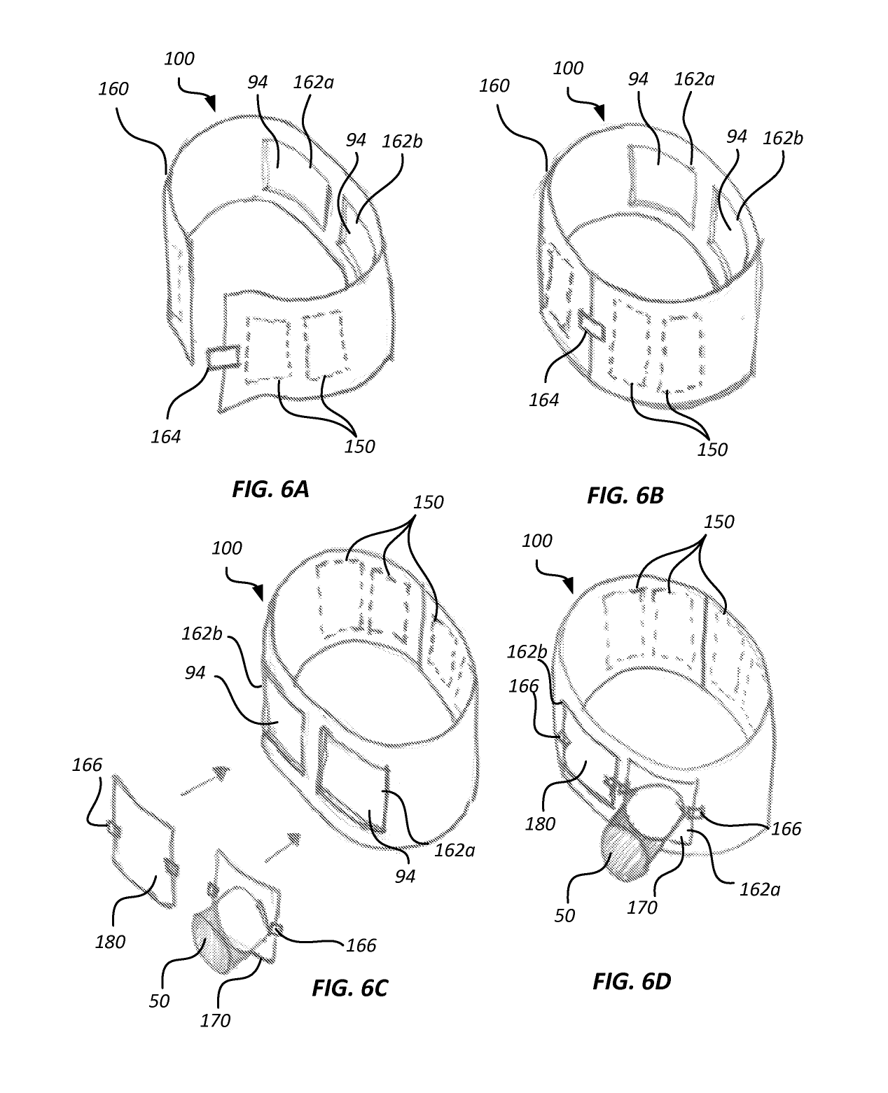

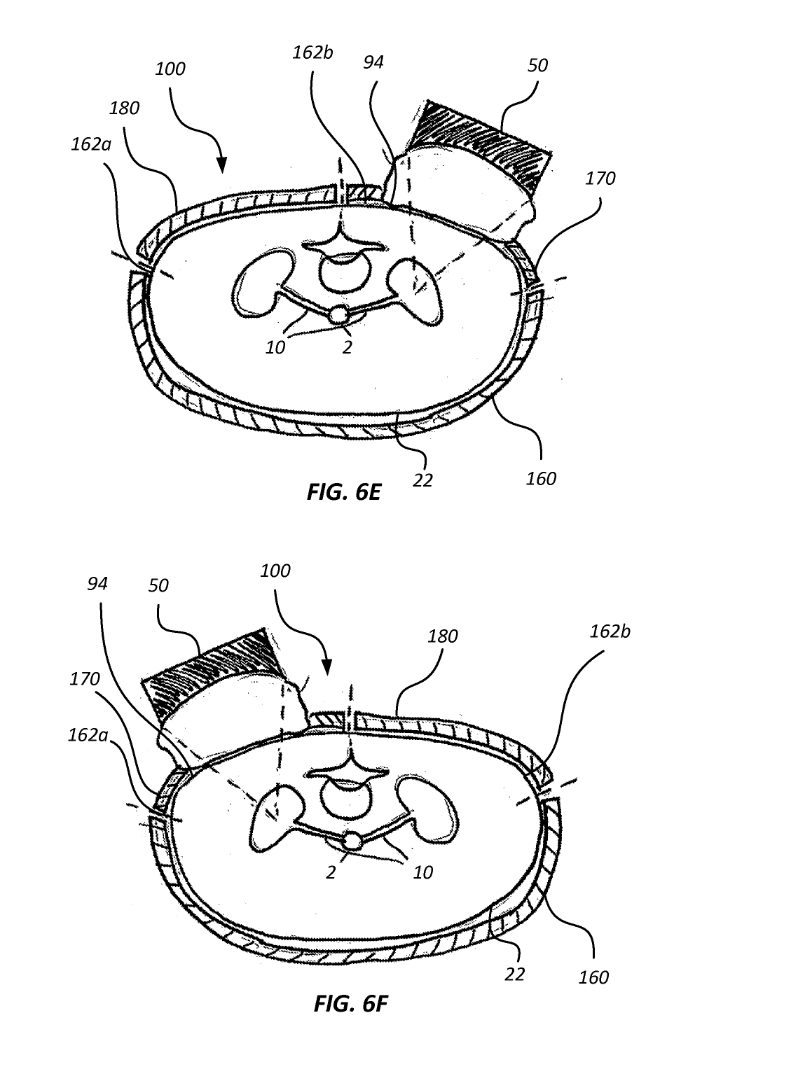

According to some embodiments, as shown in FIGS. 6A-B for example, an ablation system 100 can be provided for imaging a patient during ultrasound treatment. The ablation system 100 can include a support portion 160 configured to conform to surface anatomy of a patient, such as an abdomen. For example, the support portion 160 can be configured to wrap entirely around a circumference of a body of the patient while conforming to surface anatomy. The support portion 160 can be adjustable in size (e.g., circumferential distance) to accommodate a variety of patient sizes. Alternatively, ablation systems 100 can be provided with a variety of sizes of the support portion 160 to accommodate a variety of patient sizes. The portions of the ablation system 100 can wrap around a portion of body anatomy and secure to itself with a securing mechanism 164, as shown in FIGS. 6A-B. The securing mechanism 164 can include a latch, hook, lock, catch, Velcro, snap, toggle, button, or combinations thereof, including complementary structures on opposing sides of the support portion 160 for securement. The support portion 160 can stretch and/or flex a degree for accommodating different body sizes, shapes, and contours.

According to some embodiments, the support portion 160 can define one or more receptacles 162 (e.g., a first receptacle 162a and/or a second receptacle 162b). The receptacles 162 can be located on opposite (e.g., left or right) lateral sides of the patient. The support portion 160 defines a first receptacle 162a configured to receive an HIFU device 50 for targeting a first region on a first lateral side of the patient. The support portion 160 further defines a second receptacle 162b configured to receive the HIFU device 50 for targeting a second region on a second lateral side, opposite the first lateral side, of the patient.

According to some embodiments, as shown in FIGS. 6C-D for example, the ablation system 100 can include a treatment portion 170 configured to fit within the first receptacle 162a and/or the second receptacle 162b of the support portion 160. The treatment portion 170 can define a window 94 through which therapy may be provided by a HIFU device 50. Accordingly, the support portion 160 may be applied to the patient such that the first and second receptacles 162a,b are aligned to provide corresponding windows 94 when the treatment portion 170 is provided. The windows 94 may be a void through which direct access to the surface anatomy 22 of the patient can be achieved. Alternatively, the windows 94 may be an echolucent material through which ultrasound beams of the HIFU device 50 can be transmitted. The ablation system 100 can further include a modular portion 180 configured to fit within the first receptacle 162a and/or the second receptacle 162b of the support portion 160. The treatment portion 170 and/or the modular portion 180 can secure to the support portion 166 with a securing mechanism 166, as shown in FIGS. 6C-D. The securing mechanism 166 can include a latch, hook, lock, catch, Velcro, snap, toggle, button, or combinations thereof, including complementary structures on the support portion 160 for securement.

While the treatment portion 170 is positioned within one of the first and second receptacles 162a,b to provide HIFU therapy, the modular portion 180 can be positioned within the other of the first and second receptacles 162a,b. Subsequently, the position of the treatment portion 170 and the modular portion 180 can be swapped to provide HIFU therapy on an opposite lateral side, as shown in FIGS. 6E-F. The treatment portion 170 can be swapped at the modular portion 180 during a procedure in which the support portion 160 remains stationary relative to the patient and conforming to the surface anatomy 22.

According to some embodiments, the ablation system 100 can include one or more radiofrequency (“RF”) coils and/or RF coil arrays of an MRI device 150 embedded within a support portion 160, the treatment portion 170, and/or a modular portion 180. Radiofrequency coils can be utilized during an MRI procedure to monitor the activity and effect of a HIFU device 50. Results observed via an MRI procedure may be reported or transmitted to guide, initiate, or cease a HIFU therapy. For example, a control system governing positioning and orientation of a HIFU device 50 can be guided based on operation of an MRI device 150.

MRI systems may be used for planning surgery and/or during actual destruction of tissue. MRI systems using separate scanning sequences provide thermal level information and, in addition, also provide tissue information. Thus, the actual thermal level of the tissue can be ascertained using magnetic resonance imaging methods, and the ablation of the tissue can be observed using the MRI system.

According to some embodiments, an MRI device 150 for guiding HIFU operation comprises at least one of a coil that generates a static magnetic field, a RF coil, an x-gradient coil, a y-gradient coil, and a z-gradient coil. One or more coils allow sequences of currents to acquire PRF measurements and sequences to acquire T1 weighted images. There are several MRI methods may be used for measuring thermal levels using well-known MRI parameters, such as the spin-lattice relaxation time (“T1”). Sequence parameters—such as the time to repeat (“TR”), the time to echo (“TE”), and the flip angle—may be chosen by the user. For example, thermal level maps can be generated based on such procedures that provide T1 derived images evaluated with fast spoiled gradient echo sequences applied during the actual thermal therapy exposure. The parameters used are to some degree based on the tissue type and the precise evaluation of the behavior due to physiological or metabolic changes in the tissue during thermal therapy exposure. For example, TE, TR, and the flip angle of the spoiled gradient echo may be specified in the sequence.

The heated region may be imaged with the use of the MRI systems, employing a thermal level sensitive MR pulse sequence to acquire a thermal level “map” that is used basically to assure that the heat is being applied to the tissue and not to the surrounding healthy tissue. This is done by applying a quantity of heat that is insufficient to cause necrosis but is sufficient to raise the thermal level of the heated tissue. The MRI system thermal level map shows whether or not the heat is applied to the previously located tissue. The imaging system is also used in a separate scan sequence to create an image of the tissue intended to be destroyed. Using the imaging system in the prior art, the operator of the apparatus adjusts the placement of the radiation on the site of the tissue to be destroyed. The MR image of the tissue acquired in the separate scan determines in real time if necrosis is occurring and effectively ablating the tissue. However, the monitoring and guiding are provided using separate two-dimensional scan sequences.

Various methods for acquiring electromagnetic signals are known, in particular in the magnetic resonance imaging (MRI) field. They generally include subjecting the body to a high-intensity magnetic induction B0, typically between 0.1 and 3 Tesla. The effect of this induction is to orient the magnetic moments of the protons of the hydrogen contained in the water molecules of the body in a direction close to the main direction of the magnetic induction B0. The body part imaged is then subjected to a radiofrequency wave applied perpendicular to the magnetic induction B0 and the frequency of which is typically adjusted to the Larmor precession frequency of the hydrogen nucleus in the magnetic induction B0 in question. Immediately after the transmission of this radio frequency wave, the magnetic moments that have been subjected to the wave begin to oscillate around their equilibrium position and again take up a position along their original direction, close to that of the magnetic induction B0. During the relaxation, each water proton that has come into resonance creates, as a result, a relatively weak electromagnetic signal, called a magnetic resonance signal. This signal can then be detected by means of an appropriate detection module. Gradients of the magnetic induction B0 can be used in various spatial directions, so as to have different induction values between two points in space, each corresponding to an elementary volume of the body in question. The use of magnetic induction B0 gradients therefore allows spatial localization of the signal. The step of coding the space by means of the gradients is carried out between the proton excitation and the magnetic resonance signal reception.

In some exemplary methods, referred to as “time of flight” methods, the radio frequency waves are transmitted repeatedly and regularly, in a train of pulses. In some exemplary methods, referred to as “phase contrast” methods, takes advantage of the relationship that exists between the phase of the detected magnetic resonance signal and the rate of proton displacement in the body in question, to allow detection of blood vessels within the body. In some exemplary methods, a contrast product is injected into a body to enhance an image.

Various MRI methods may be used for measuring thermal levels using well-known MRI parameters, such as the spin-lattice relaxation time (“T1”). Sequence parameters—such as the time to repeat (“TR”), the time to echo (“TE”), and the flip angle—may be chosen by the user. For example, thermal level maps can be generated based on such procedures that provide T1 derived images evaluated with fast spoiled gradient echo sequences applied during the actual thermal therapy exposure. The parameters used are to some degree based on the tissue type and the precise evaluation of the behavior due to physiological or metabolic changes in the tissue during thermal therapy exposure. For example, TE, TR, and the flip angle of the spoiled gradient echo may be specified in the sequence. Sequence parameters may be used to localize the low-thermal level elevation induced by a focused ultrasound beam during both the planning and treatment.

According to some embodiments, magnetic resonance (MR) thermometry can be based on proton resonance frequency (PRF) shift to monitor temperature changes in an area heated by HIFU in MRI-guided HIFU equipment, further based on the phenomenon of the resonance frequency of the protons in water being offset (shifted) dependent on the temperature change. MR thermometry based on PRF-shift requires that a base image (MR phase image) before heating, also referred to as a reference image, be generated, with the reference image providing information on a reference phase. By subtraction from the phase image (also referred to as a heated image) acquired during heating or after heating, the exact value of the elevated temperature in the heated area can be determined.

As used herein, “thermal level” includes absolute temperature, relative temperature, temperature change, heat, change in heat, relative heat, thermal dosage, and other metrics related to thermal conditions.

Incorporated herein by reference are the following US applications and/or publications containing further teachings regarding MR imaging: US Pub. No. 2009/0275821, published on May 5, 2008; US Pub. No. 2006/0058642, published on Mar. 16, 2006; US Pub. No. 2010/0217114, published on Aug. 26, 2010.

According to some embodiments, application of focused sound energy to points around an artery has the result that sympathetic nerves are damaged and the artery is not damaged. The temperature of the artery may be substantially maintained by blood flow through the artery during the procedure while temperature of at least one nerve is elevated. Tissue near the nerves and the artery may be monitored by MRI or other means, whereby delivery of heat may be ceased when a thermal level exceeds a threshold.

According to some embodiments, a cooling catheter may be provided within the artery in a vicinity of the focal region of the focused sound energy. The cooling catheter provides maintenance of reduction of thermal levels in or around the artery to reduce or eliminate damage to the artery. According to some embodiments, a catheter may be provided at, along, or aligned with a target location within an artery. The catheter may provide a localizing signal to an MRI scanner or other device to identify the target location. The target location may identify where a focal region of the focused sound energy should be applied.

According to some embodiments, the method includes, as a result of the heating, lowering a blood pressure in a mammal. According to some embodiments, devices and methods disclosed herein may be used to treat Congestive Heart Failure (“CHF”) or related conditions, including hypertension. In addition to their role in the progression of CHF, the kidneys play a significant role in the progression of Chronic Renal Failure (“CRF”), End-Stage Renal Disease (“ESRD”), hypertension (pathologically high blood pressure) and other cardio-renal diseases. The functions of the kidneys can be summarized under three broad categories: filtering blood and excreting waste products generated by the body's metabolism; regulating salt, water, electrolyte, and acid-base balance; and secreting hormones to maintain vital organ blood flow. Without properly functioning kidneys, a patient will suffer water retention, reduced urine flow and an accumulation of waste toxins in the blood and body. These conditions result from reduced renal function or renal failure (kidney failure) and are believed to increase the workload of the heart. In a CHF patient, renal failure will cause the heart to deteriorate further as fluids are retained and blood toxins accumulate due to the poorly functioning kidneys.

It has been established in animal models that the heart failure condition results in abnormally high sympathetic activation of the kidneys. An increase in renal sympathetic nerve activity leads to decreased removal of water and sodium from the body, as well as increased renin secretion. Increased renin secretion leads to vasoconstriction of blood vessels supplying the kidneys, which causes decreased renal blood flow. Reduction of sympathetic renal nerve activity, e.g., via renal nerve ablation, may reverse or ameliorate processes.

According to embodiments, heating may be ceased for a period of time between any ablation procedure and a subsequent procedure on ipsilateral renal nerves. For example, a time period may be sufficient to allow inflammation to recede, scar tissue to begin forming, blood pressure to equilibrate, and any compensatory hypertensive effect from the contralateral kidney to manifest. For example, the time period may be greater or less than 1 day, 10 days, 100 days, and 1000 days. By further example, the time period may be equal to or greater than 1, 2, 3, 4, 5, 6, 7, 10, 15, 30, 60, 90, 120, or 180 days. By further example, the time period may be equal to or greater than 1, 2, 3, 4, 5, 6, 7, 8, 9, 10, 11, 12, 18, or 24 months.

According to some embodiments, devices and methods disclosed herein may be used in conjunction or combination with other devices and methods for achieving renal neuromodulation, including localized drug delivery (such as by a drug pump or infusion catheter), stimulation electric field, and laser therapy, inter alia.

Incorporated herein by reference are the following US applications and/or publications containing teachings regarding renal nerve ablation techniques: US Pub. No. 2010/0057150, published on Mar. 4, 2010; US Pub. No. 2010/0222854, published on Sep. 2, 2010; US Pub. No. 2008/0213331, published on Sep. 4, 2008.

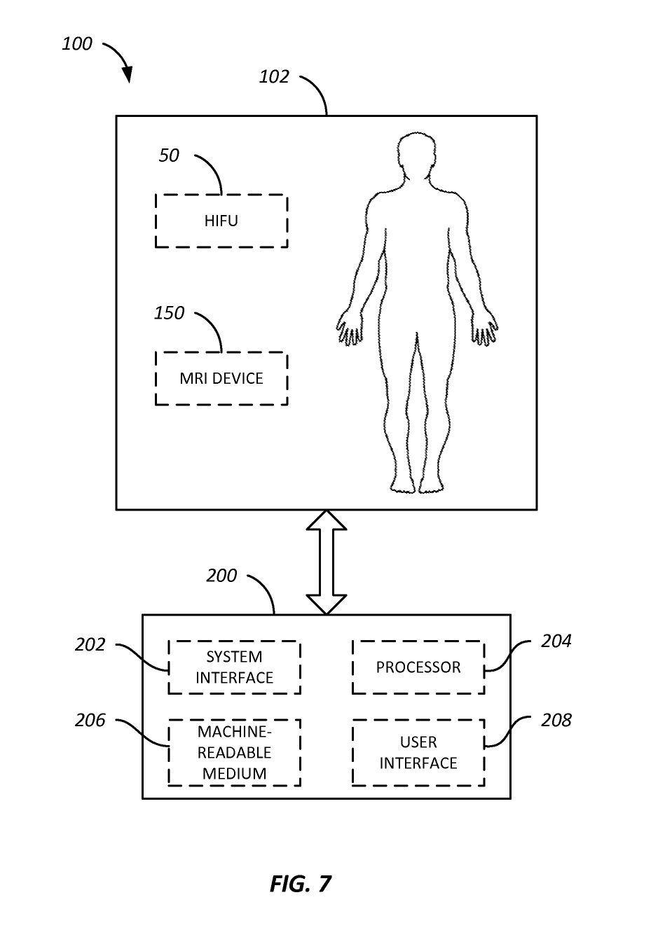

According to some embodiments, as shown in FIG. 7, an ablation system 100 may comprise an operation system 102 and a control system 200. Operation system 102 may comprise a HIFU device 50 and an MRI device 150 for performing operations on a patient. According to some embodiments, a control system 200 is provided to control, monitor, or interact with one or more components of operation system 102, such as HIFU device 50 and MRI device 150.

According to some embodiments, as shown in FIG. 7, a control system 200 may comprise a system interface 202, a processor 204, a machine-readable medium 206, a user interface 208, and other components as appropriate to produce the desired functionalities of the control system 200.

The control system 200 may include a processor 204 for executing instructions and may further include a machine-readable medium 206, such as a volatile or non-volatile memory, for storing data and/or instructions for software programs. The instructions, which may be stored in a machine-readable medium 206, may be executed by the control system 200 to control and manage access to the various networks, as well as provide other communication and processing functions. The instructions may also include instructions executed by the control system 200 for various user interface devices, such as a display and a keypad. The control system 200 may include an input port and an output port. Each of the input port and the output port may include one or more ports. The input port and the output port may be the same port (e.g., a bi-directional port) or may be different ports.

The system 200 can include, in addition to hardware, code that creates an execution environment for the computer program in question, e.g., code that constitutes processor firmware, a protocol stack, a database management system, an operating system, or a combination of one or more of them stored in an included memory 204, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, and/or any other suitable storage device, for storing information and instructions to be executed by the processor 204. The processor 204 and the medium 206 can be supplemented by, or incorporated in, special purpose logic circuitry.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

As used herein, a “processor” can include one or more processors, and a “module” can include one or more modules.

In an aspect of the subject technology, a machine-readable medium is a computer-readable medium encoded or stored with instructions and is a computing element, which defines structural and functional relationships between the instructions and the rest of the system, which permit the instructions' functionality to be realized. Instructions may be executable, for example, by a system or by a processor of the system. Instructions can be, for example, a computer program including code. A machine-readable medium may comprise one or more media.

The control system 200 may be implemented using software, hardware, or a combination of both. By way of example, the control system 200 may be implemented with one or more processors. A processor may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device that can perform calculations or other manipulations of information.

A machine-readable medium can be one or more machine-readable media. Software shall be construed broadly to mean instructions, data, or any combination thereof, whether referred to as software, firmware, middleware, microcode, hardware description language, or otherwise. Instructions may include code (e.g., in source code format, binary code format, executable code format, or any other suitable format of code).

Machine-readable media may include storage integrated into a processing system, such as might be the case with an ASIC. Machine-readable media may also include storage external to a processing system, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device. Those skilled in the art will recognize how best to implement the described functionality for the control system 200. According to one aspect of the disclosure, a machine-readable medium is a computer-readable medium encoded or stored with instructions and is a computing element, which defines structural and functional interrelationships between the instructions and the rest of the system, which permit the instructions' functionality to be realized. In one aspect, a machine-readable medium is a non-transitory machine-readable medium, a machine-readable storage medium, or a non-transitory machine-readable storage medium. In one aspect, a computer-readable medium is a non-transitory computer-readable medium, a computer-readable storage medium, or a non-transitory computer-readable storage medium. Instructions may be executable, for example, by a client device or server or by a processing system of a client device or server. Instructions can be, for example, a computer program including code.

An interface (e.g., 202 and/or 208) may be any type of interface and may reside between any of the components shown in FIG. 7. An interface may also be, for example, an interface to the outside world (e.g., an Internet network interface). A transceiver block may represent one or more transceivers, and each transceiver may include a receiver and a transmitter. A functionality implemented in a control system 200 may be implemented in a portion of a receiver, a portion of a transmitter, a portion of a machine-readable medium, a portion of a display, a portion of a keypad, or a portion of an interface, and vice versa.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. An apparatus for imaging a patient during an ultrasound treatment, comprising:
a support portion configured to conform to a surface anatomy of a patient and defining a first window configured to receive an ultrasound transducer for targeting a first region on a first side of the patient when received within the first window; and
radiofrequency coil arrays attached to the support portion.

2. The apparatus of claim 1, wherein the support portion further defines a second window configured to receive the ultrasound transducer for targeting a second region on a second side, opposite the first side, of the patient.

3. The apparatus of claim 2, further comprising a modular insert configured to fit within the first window and/or the second window.

4. The apparatus of claim 2, wherein the first side is a first lateral side and wherein the second side is a second lateral side.

5. The apparatus of claim 2, wherein the first side is an anterior side and wherein the second side is a posterior side.

6. The apparatus of claim 1, wherein the support portion comprises:
a support portion defining a first receptacle on the first side and a second receptacle on the second side, opposite the first side, of the patient;
a treatment portion, defining the first window, configured to be placed in the first receptacle; and
a modular portion configured to be placed in the second receptacle.

7. The apparatus of claim 6, wherein, during a procedure in which the support portion remains stationary relative to the patient, the treatment portion is configured to be moved to the second receptacle and the modular portion is configured to be moved to the first receptacle.

8. The apparatus of claim 6, the radiofrequency coil arrays are distributed among the support portion, the treatment portion, and the modular portion.

9. The apparatus of claim 1, wherein the support portion is configured to wrap entirely around a circumference of a body of the patient while conforming to the surface anatomy.

10. The apparatus of claim 1, wherein the first window is configured to be aligned on an anterior side of the patient when the support portion conforms to the surface anatomy.

11. The apparatus of claim 1, wherein the first window is configured to be aligned on a posterior side of the patient when the support portion conforms to the surface anatomy.

12. The apparatus of claim 1, wherein the first window comprises an echolucent material.

13. The apparatus of claim 1, wherein the surface anatomy is an abdomen of the patient, the first region includes a first renal nerve of the patient, and the second region includes a second renal nerve of the patient.

14. The apparatus of claim 1, further comprising the ultrasound transducer.

15. A method of imaging a patient during an ultrasound treatment, comprising:
applying a support portion to conform to a surface anatomy of a patient;
aligning a focal region of an ultrasound transducer with a first region on a first side of the patient;
ablating tissue within the first region with ultrasound energy from the transducer and through a first window of the support portion; and
obtaining an image of the first region with radiofrequency coil arrays attached to the support portion.

16. The method of claim 15, further comprising:
aligning the focal region of the ultrasound transducer with a second region on a second side of the patient;
ablating tissue within the second region with ultrasound energy from the transducer and through a second window of the support portion; and
obtaining an image of the second region with the radiofrequency coil arrays.

17. The method of claim 16, further comprising placing a modular portion, containing some of the radiofrequency coil arrays, in the second window.

18. The method of claim 16, further comprising placing a modular portion, containing some of the radiofrequency coil arrays, in the first window.

19. The apparatus of claim 16, wherein the surface anatomy is an abdomen of the patient, the first region includes a first renal nerve of the patient, and the second region includes a second renal nerve of the patient.

20. The method of claim 15, wherein the support portion remains stationary relative to the patient during the aligning.

21. The method of claim 15, wherein the applying comprises aligning the first window on an anterior side of the patient.

22. The method of claim 15, wherein the applying comprises aligning the first window on a posterior side of the patient.

23. An apparatus for performing an ultrasound treatment, comprising:
an ultrasound transducer having a focal length at which a focal region is formed; and
an expandable reservoir comprising:
a first surface configured to conform to a surface anatomy of a patient; and
a port in fluid communication with a fluid source;
wherein the expandable reservoir is configured to change a size in a dimension by inflating or deflating the expandable reservoir.

24. The apparatus of claim 23, wherein the size of the expandable reservoir determines a location of the focal region within the patient when the expandable reservoir conforms to the surface anatomy.

25. The apparatus of claim 23, wherein the expandable reservoir abuts the ultrasound transducer.

26. The apparatus of claim 23, wherein the focal length is fixed relative to the transducer.

27. A method for performing an ultrasound treatment, comprising:
applying a first surface of an expandable reservoir to a patient to conform to a surface anatomy of the patient;
positioning, within the patient, a focal region of an ultrasound transducer connected to the expandable reservoir by inflating or deflating the expandable reservoir.

28. The method of claim 27, wherein the positioning comprises injecting fluid into the expandable reservoir, aspirating fluid out of the expandable reservoir, or both.

29. The apparatus of claim 27, wherein the surface anatomy is an abdomen of the patient and the positioning comprises aligning the focal region with a renal nerve of the patient.
